(19) United States
(12) Patent Application Publication
(10) Pub. No.: US 2007/0122459 A1
(43) Pub. Date: May 31, 2007

(54) Composition and Method for Preventing or Treating Urolithiasis

(76) Inventor: Shiguang Yu, Topeka, KS (US)

Correspondence Address: COLGATE-PALMOLIVE COMPANY, 909 RIVER ROAD, PISCATAWAY, NJ, 08855, US

(21) Appl. No.: 11/564,681

(22) Filed: Nov. 29, 2006

Related U.S. Application Data
(60) Provisional application No. 60/740,560, filed on Nov. 29, 2005.

(51) Int. Cl. A61K 33/14; A23K 1/165

(52) U.S. Cl. 424/442; 424/679

(57) ABSTRACT

A method for preventing or treating urolithiasis in an animal by feeding the animal a food composition comprising potassium in an amount equivalent to more than about 1% by weight potassium chloride and a food composition suitable for preventing or treating urolithiasis in an animal comprising potassium in an amount equivalent to more than about 1% by weight potassium chloride.

### CROSS-REFERENCE TO RELATED APPLICATIONS

This application claims priority to U.S. Provisional Application Ser. No. 60/740,560 filed Nov. 29, 2005, the disclosure of which is incorporated herein by reference.

### BACKGROUND OF THE INVENTION

1. Field of the Invention

This invention relates generally to food compositions for animal consumption and particularly to food compositions and methods for using food compositions for preventing or treating urolithiasis.

2. Description of the Related Art

Urolithiasis is a urinary tract disorder characterized by presence of solid deposits such as uroliths or urinary calculi (also known as stones) or excessive amounts of crystals in the urinary tract. These solid deposits can form anywhere in the urinary system, including the kidneys, ureters, urethra or, most commonly, the bladder. The deposits can irritate the lining of the urinary tract, cause changes in the lining, and often cause pain. In extreme cases, uroliths can partially or totally block the flow of urine and cause urination to become difficult, painful, or impossible.

Urolith formation is a significant clinical problem for many animals, including companion animals such as dogs and cats. There are many types of uroliths such as struvite, calcium oxalate, calcium phosphate, ammonium acid urate, carbonate apatite, xanthine, cystine, and silica. Two prevalent types of uroliths in cats and dogs are struvite, also known as MAP (magnesium ammonium phosphate), and calcium oxalate.

Although the etiology of urolithiasis in animals is not fully understood, many factors may be involved in the formation of various types of uroliths in animals. These factors include, but are not limited to, oversaturated urine, extreme urine pH, urinary tract bacteria infection, genetic makeup, age, sex, and dietary factors.

Methods for treatment of urolithiasis have focused on surgical removal of uroliths or, in the case of struvite uroliths, dietary adjustments. Methods also include using drugs that result in an acidic urine to dissolve the urolith. Such dietary adjustments typically involve the modification of food so that acidic urine is produced after the food is metabolized in the body. Often, the dietary adjustments also have restricted amounts of magnesium, phosphorus, and protein (protein restriction leads to decreased ammonium levels) and in some cases increased levels of sodium (generally as sodium chloride) which can stimulate water consumption by the animal. These dietary adjustments have inherent disadvantages. For example, an animal must be limited more or less completely to a specific food. Otherwise the crucial restriction of magnesium, phosphorus, and protein can easily be undone. Further, increased sodium intake may have deleterious effects on the animal's long-term health in certain conditions such as in animals with kidney disease.

Similarly, for calcium oxalate uroliths, reduced dietary intake of oxalate, calcium, sodium, and protein are typically recommended. Such dietary adjustments are disadvantageous, however, because reduction of intake of essential minerals can be detrimental to the animal's health.

Prevention of urolith formation has also focused on increasing water intake to increase urine volume and therefore reduce the concentration of urolith-forming components. Therefore, increasing water intake is the most common recommendation in the management of urolithiasis in animals as well as in humans. Increasing urine volume, however, can be difficult in animals. For instance, for companion animals such as dogs and cats, veterinarians typically recommend replacing of dry foods with high moisture foods such as calmed pet foods to increase water intake and urine volume. A dietary approach to preventing and treating that permits feeding wet or dry foods would be a desirable advance in the art.

### SUMMARY OF THE INVENTION

The invention provides a food composition useful for preventing or treating urolithiasis in an animal comprising potassium in an amount equivalent to more than about 1% by weight potassium chloride on a dry matter basis. Typically, the composition is nutritionally adapted for feeding to an animal as a substantial part of the animal's diet.

The invention also provides a method for preventing or treating urolithiasis in an animal comprising feeding the animal a food composition comprising potassium in an amount equivalent to more than about 1% by weight potassium chloride. Generally, the food composition is feed as a substantial part of the animal's diet for a period sufficient to prevent or treat urolithiasis.

The invention further provides a method for promoting urinary wellness in an animal comprising feeding the animal a food composition comprising potassium in an amount equivalent to more than about 1% by weight potassium chloride.

The invention also provides a kit comprising a potassium supplement and one or more of (a) a base food nutritionally adapted for feeding to an animal as a substantial part of its diet; (b) an anti-urolithiasis agent other than a potassium supplement; (c) a diagnostic device for measuring urine pH; and (d) a means for communicating information about or instructions for one or more of (i) using the diagnostic device; (ii) adjusting or controlling urine pH; (iii) adding the potassium supplement to the base food to provide a food composition comprising potassium in an amount equivalent to more than about 1% by weight potassium chloride; and (iv) promoting urinary wellness or preventing or treating urolithiasis in an animal.

The invention further provides a means for communicating information about promoting urinary wellness or preventing or treating urolithiasis in an animal by feeding the animal a food composition comprising potassium in an amount equivalent to more than about 1% by weight potassium chloride. Such communicating means can illustratively be selected from the group consisting of product labels, package inserts, brochures, handouts, advertisements, public announcements, audiotapes, videotapes, DVDs, CD-ROMs, computer readable chips, cards and disks, computer memory, web pages, and combinations thereof.

### DETAILED DESCRIPTION OF THE INVENTION

The invention provides a composition suitable for preventing or treating urolithiasis in an animal. The composition comprises a urolithiasis preventing or treating amount of potassium. The invention is based upon the discovery that feeding an animal a potassium chloride-enriched food composition increases urine volume. Increasing urine volume prevents urolithiasis in animals susceptible to the disorder or treats urolithiasis in animals suffering from the disorder. Generally, the potassium chloride-enriched food composition is feed to an animal as part of a substantially complete diet for a period sufficient to prevent or treat the disorder.

The invention is effective when the food composition is a dry food, e.g., kibbles. The effect has been observed in both canines and felines. An increase in urine volume is useful for preventing and/or treating urolithiasis and therefore in promoting urinary wellness of an animal.

A “potassium-enriched” food herein is one that is nutritionally adapted for feeding to an animal as a substantial part of its diet, but contains a higher level of potassium than is typically recommended for feeding a healthy animal.

Feeding a food composition as “a substantial part” of an animal's diet means that the food composition provides a sufficient daily ration to satisfy the animal's dietary requirements at least of energy and protein. Supplements and/or treats can be consumed by the animal in addition to such a food composition. The particular potassium-enriched food composition can be varied over time, but the potassium level should remain above the typically recommended level for a healthy animal, for a period effective to prevent or treat urolithiasis.

The term “animal” means any animal susceptible to or suffering from urolithiasis. The methods and compositions of the invention are useful for a variety of human and non-human animals susceptible to or suffering from urolithiasis, including avian, bovine, canine, equine, feline, hicrine, murine, ovine, and porcine animals. In some embodiments, the animal is a companion animal such as canine or feline, particularly a dog or a cat.

In a particular embodiment, the animal is a companion animal. A “companion animal” herein is an individual animal kept by a human caregiver as a pet, or any individual animal that has been widely domesticated as pets, including dogs (Canis familiaris) and cats (Felis domesticus), whether or not the individual animal is kept solely or partly for companionship. Thus “companion animals” herein include working dogs, farm cats kept for rodent control, etc., as well as pet dogs and cats.

Foods nutritionally adapted for canines and felines (herein referred to as “pet foods”) have heretofore typically contained potassium in an amount equivalent to up to about 1% potassium chloride (KCl) on a dry matter basis, in many cases much less than this. It is believed, in accordance with the invention, that any increase in the amount of potassium in an animal's diet can be helpful in preventing or treating urolithiasis, for example by increasing urine volume. Accordingly, a food composition of the present invention comprises potassium in a KCl equivalent amount greater than about 1%.

The amount of potassium in the composition does not exceed a level that is safe and non-toxic to the animal. For example, the potassium level should not be so high as to cause hyperkalemia. In one embodiment the KCl equivalent amount of potassium is more than about 1%, up to about 20%.

More typically, the KCl equivalent amount of potassium in the composition is about 2% to about 8%, for example about 3% to about 5%. For both canines and felines, a potassium level of at least about 2% will generally be found suitable, and further benefits can be obtained, particularly in felines, by increasing this to at least about 3%, again expressed as KCl equivalent.

Potassium can be present in a food compositions of the invention in any form, but is generally in the form of a potassium salt, including without limitation acetate, bicarbonate, chloride, citrate, cyanate, gluconate, glutamate, glycerophosphate, guaiacol sulfonate, iodate, nitrite, phosphate, sorbate, sulfate and tartrate salts and combinations thereof. In one embodiment the potassium is present at least in part as one or more salts selected from the group consisting of potassium chloride, potassium citrate, potassium sulfate and combinations thereof. Most commonly the potassium is present wholly or partly as potassium chloride; it is for this reason that potassium amounts herein are expressed as potassium chloride (KCl) equivalent amounts. KCl equivalent amounts can be converted to potassium ion amounts using a conversion factor of about 0.52.

Potassium-enriched wet or dry foods can be prepared by any conventional process for such foods, wherein one or more potassium salts, most typically potassium chloride, is added to the mix or blend of ingredients. It is generally most convenient to add the potassium salt in aqueous solution to ensure homogeneous distribution in the resulting food composition.

In an embodiment of the invention, a method is provided for increasing urine volume in an animal, for example a canine or feline. This method comprises feeding the animal a potassium-enriched food composition as described above.

Without being bound by theory, it is believed that feeding a potassium chloride-enriched food composition to an animal leads to an increase in osmolarity of the animal's intercellular fluid. Such increased osmolarity tends to make the animal thirstier, leading to an increase in water consumption. As part of its water regulation function, the animal's body then diverts a substantial portion of this increased water consumption to the urine, increasing its total volume. An increase in urine volume is normally associated with a decrease in urine specific gravity, as the urine becomes less concentrated in solutes including the minerals that tend to form uroliths.

The present invention provides a method for preventing or treating urolithiasis in an animal. The method comprises feeding the animal a potassium-enriched food composition as described above. Generally, the composition is feed as a substantial part of the animal's diet for a urolithiasis preventing or treating effective period. What constitutes a urolithiasis preventing or treating effective period depends on a number of factors, including the species and breeds of the animal, any disease conditions that are predisposing to urolith formation (e.g., bacterial infection of the urinary tract), the degree of potassium enrichment of the food composition, whether or not uroliths are already present, and other factors. A benefit in urine volume and/or urine specific gravity can occur within days (e.g., within about 5 days) of commencement of feeding the composition. In particular situations, a feeding period effective to prevent or treat urolithiasis can be as little as about 5 days, or can be longer, for example at least about 1 week, at least about 2 weeks, at least about 4 weeks, at least about 6 weeks or at least about 12 weeks. For an animal chronically at risk of urolithiasis, a food composition of the invention can be fed as a substantial part of its diet for an unlimited period, for example for the duration of the animal's life.

In management of urolithiasis or risk thereof, a method of the invention can be used alone or in combination with one or more other approaches. Such approaches include dietary intervention in addition to potassium enrichment, for example adjustment in diet to promote a desirable urine pH. In one embodiment, the present method comprises, in addition to feeding a potassium-enriched food composition as described herein, administering one or more anti-urolithiasis agents.

An “anti-urolithiasis agent” herein is an agent (for example a drug or a nutraceutical) having preventive or therapeutic anti-urolithiasis effect by any mechanism, for example by cooperating with the potassium-enriched food to increase urine volume, or, cooperatively with or independently of the potassium-enriched food, by modifying urine pH, or preventing or treating a bacterial infection of the urinary tract such as antibiotics.

As urine pH is a readily measured and useful indicator of urolithiasis risk, the present method optionally further comprises monitoring the animal's urine pH during the feeding period. Any known device for measuring urine pH, including indicator strips or dipstick, can be used.

Urine pH can be influenced by certain components of the food composition. For example, cation-anion balance can have an effect, as can levels of sulfur-containing amino acids. In one embodiment, the food composition comprises (a) potassium in an amount equivalent to more than about 1% by weight potassium chloride; and one or both of (b) cations and anions in a ratio effective to produce a desirable urine pH; and (c) sulfur-containing amino acids in an amount effective to produce a desirable urine pH.

Urolithiasis is one of several conditions that can be mediated by low water intake that leads to low urine volume and corresponding high urine specific gravity. Thus, in another embodiment, a method is provided for preventing or treating a low water intake mediated condition in an animal, comprising feeding the animal, as a substantial part of its diet for a period effective for such prevention or treatment, a potassium-enriched food composition as described above.

Urinary tract infections, for example bacterial infections, can be ameliorated by increasing water intake and hence urine volume. In one embodiment, therefore, a method is provided for preventing or treating a urinary tract infection in an animal, comprising feeding the animal, as a substantial part of its diet for a period effective for such prevention or treatment, a potassium-enriched food composition as described above. The method optionally further comprises administering an antibacterial drug such as antibiotics.

According to any of the above methods, the potassium-enriched food can be pre-manufactured or prepared by the animal's caregiver, for example immediately before feeding. Preparation by the caregiver can be by adding a potassium supplement, for example a supplement comprising a potassium salt such as potassium chloride, to a base food in an amount sufficient to raise the potassium content of the resulting composition above about 1% KCl equivalent. The base food and the potassium supplement can be sourced or purchased independently of one another, but conveniently for the caregiver these items can be supplied in a kit.

In one aspect, the invention provides a kit comprising a potassium supplement and one or more of the following:
(a) a base food nutritionally adapted for feeding to an animal as a substantial part of its diet;
(b) an anti-urolithiasis agent other than a potassium supplement;
(c) a diagnostic device for measuring urine pH; and
(d) a means for communicating information about or instructions for one or more of the following:
   (i) using the diagnostic device;
   (ii) adjusting or controlling urine pH;
   (iii) adding the potassium supplement to the base food to provide a food composition comprising potassium in an amount equivalent to more than about 1% by weight potassium chloride; and
   (iv) promoting urinary wellness or preventing or treating urolithiasis in an animal.

The kit components can be physically bundled or packaged together or can be provided in a virtual package. A “virtual package” is a means for providing the elements of a kit to a consumer, in this case an animal's caregiver, wherein the elements are not physically bundled or packaged together. Examples of virtual packaging are well known in the marketing arts. For example, a package containing the potassium supplement can have, for example, on wrapping or a label on the package or on a package insert, a coupon for purchase of the base food (or vice versa). Alternatively, such a coupon could be for purchase of an anti-urolithiasis agent or a diagnostic device for measuring urine pH. The coupon optionally entitles the purchaser to a discount or rebate on the purchase price of one or more elements of the kit. As another example of a virtual package, the potassium supplement, the base food, the anti-urolithiasis agent or the diagnostic device can carry a reference to a communicating means, for example information about or instructions for use of the kit, located elsewhere, for example in a virtual environment such as a website.

In another aspect, the invention provides a means for communicating information about or instructions for promoting urinary wellness or preventing or treating urolithiasis in an animal by feeding the animal a food composition comprising potassium in an amount equivalent to more than about 1% by weight potassium chloride. The communicating means can utilize any medium or combination of media, including printed media (documents), digital or optical storage media, audiovisual media, etc., and can illustratively be selected from the group consisting of product labels, package inserts, brochures, handouts, advertisements, visual displays, public announcements, audiotapes, videotapes, DVDs, CD-ROMs, computer readable chips, cards and disks, computer memory, web pages, and combinations thereof. Preferred communicating means include a product label, a package insert, a brochure, an advertisement, a visual display or a website, for example accessible via a user interface at a point of sale.

In a further aspect, the present invention provides for a use of a composition comprising potassium in an amount equivalent to more than about 1% by weight potassium chloride to prepare a medicament. In another, the invention provides for the use of such composition to prepare a medicament for maintaining and/or improving animal health, e.g., for preventing or treating urolithiasis in an animal. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

All percentages expressed herein are on a weight by dry matter basis unless specifically stated otherwise.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

### EXAMPLES

The invention can be further illustrated by the following examples of preferred embodiments thereof, although it will be understood that these examples are included merely for purposes of illustration and are not intended to limit the scope of the invention unless otherwise specifically indicated.

### Example 1

A study was conducted using a Latin-Square design to investigate effect of increasing dietary potassium chloride on water intake and various urine parameters including urine volume and urine specific gravity in canines.

Nine beagles were randomly divided into 3 groups, 3 dogs in each group. All dogs were housed individually so that urine from each dog could be independently collected. The daily amount of food provided to each dog was adjusted for maintenance of body weight. Deionized water was available to the dogs at all times.

Dogs were fed their assigned foods according to the Latin-Square design for 10 days. The control food (Food 1) was a dry commercial canine food composition comprising yellow corn, poultry by-product meal, soybean meal, corn gluten meal, soybean mill run, compacted egg, calcium sulfate dihydrate, whole flaxseed, iodized salt, taurine, choline chloride, potassium chloride (about 0.2% by weight of the food), mineral premix, vitamin premix, animal fat, soybean oil, and palatability enhancers. The test food (Food 2) was made by supplementing Food 1 with potassium chloride (about 2.3% by weight of the food). No other food was provided during the study period.

Food and water intake and urine volume were recorded for each dog. Urine was collected from all dogs during the last 5 days of the test period, and analyzed for volume, specific gravity, phosphorus (P), magnesium (Mg), calcium (Ca), sodium (Na), chloride (Cl) and potassium (K). Results, averaged for all dogs, are presented in Table 1.

TABLE 1
Results of feeding study comparing Foods 1 and 2 in canines

| Parameter | Food 1 | Food 2 |
|---|---|---|
| Food intake (g/day) | 214 | 223 |
| Water intake (g/day) | 328 | 446 |
| Water intake/food intake | 1.52 | 2.00 |
| Urine volume (g/day) | 213 | 305 |
| Urine specific gravity | 1.028 | 1.024 |
| Urine P (ppm) | 1181 | 694 |
| Urine Mg (ppm) | 93 | 74 |
| Urine Ca (ppm) | 59 | 49 |
| Urine Na (ppm) | 1646 | 1209 |
| Urine Cl (mM) | 97 | 197 |
| Urine K (ppm) | 4317 | 8151 |

After consuming the potassium chloride-enriched food for 10 days, the dogs exhibited a 36% increase in water intake, a 32% increase in ratio of water intake to food intake, and a 43% increase in urine volume. Urine specific gravity was reduced from 1.028 to 1.024. Concentrations of P, Mg, Na and Ca in urine were also reduced.

In summary, this study showed that factors predisposing to urolithiasis in canines, including low urine volume and high urine specific gravity, could be ameliorated by feeding a potassium chloride-enriched food composition according to the invention.

### Example 2

A study was conducted using a Latin-Square design to investigate effect of increasing dietary potassium chloride on water intake and various urine parameters including urine volume and urine specific gravity in felines.

Nine American domestic shorthair cats were randomly divided into 3 groups, 3 cats in each group. All cats were housed individually so that urine from each cat could be independently collected. The daily amount of food provided to each cat was adjusted for maintenance of body weight. Deionized water was available to the cats at all times.

Cats were fed their assigned foods according to the Latin-Square design for two weeks. The control food (Food 3) was a dry commercial feline food composition comprising brewers rice, poultry by-product meal, corn gluten meal, calcium sulfate dihydrate, choline chloride, calcium carbonate, iodized salt, taurine, vitamin premix, mineral premix, animal fat, palatability enhancers, and potassium chloride (about 1.0% by weight of the food). Test foods were made by supplementing Food 3 with additional potassium chloride (about 2.3% by weight of the food, Food 4; or about 3.6% by weight of the food, Food 5). No other food was provided during the study period.

Food and water intake and urine volume were recorded for each cat. Urine were collected from all cats during the second week of the study, and analyzed for urine volume, specific gravity, pH, P, Mg, Ca, Na, Cl and K. Results, averaged for all cats, are presented in Table 2.

TABLE 2
Results of feeding study comparing Foods 3, 4 and 5 in felines

| Parameter | Food 3 | Food 4 | Food 5 |
|---|---|---|---|
| Intake of KCl, g/day | 0.42 | 0.88 | 1.53 |
| Food intake, g/day | 43.7 | 43.3 | 42.9 |
| Water intake, g/day | 80 | 87 | 106 |
| Water intake/food intake | 1.84 | 1.99 | 2.44 |
| Urine volume, g/day | 37.0 | 46.5 | 53.1 |
| Urine volume/food intake | 0.85 | 1.07 | 1.24 |
| Urine specific gravity | 1.057 | 1.050 | 1.043 |
| Urine pH | 6.64 | 6.73 | 6.96 |
| Intake of KCl, g/day | 0.42 | 0.88 | 1.53 |
| Urine P (ppm) | 2148 | 1754 | 1409 |
| Urine Mg (ppm) | 82 | 62 | 50 |
| Urine Ca (ppm) | 54 | 54 | 55 |
| Urine Na (ppm) | 3552 | 3059 | 2361 |
| Urine Cl (ppm) | 10777 | 13081 | 13755 |
| Urine K (ppm) | 9686 | 13211 | 14611 |

After consuming Food 4, the cats exhibited increases in water intake, in ratio of water intake to food intake, and in urine volume. Urine specific gravity was reduced from 1.057 to 1.050. Concentrations of P, Mg and Na in urine were also reduced. After consuming Food 5, water intake, ratio of water intake to food intake, and urine volume were all further increased, urine specific gravity further reduced to 1.043, and concentrations of P, Mg and Na in urine further reduced.

In summary, this study showed that factors predisposing to urolithiasis in felines, including low urine volume and high urine specific gravity, could be ameliorated by feeding a potassium chloride-enriched food composition according to the invention.

### Example 3

A study was conducted to investigate effect of increasing dietary potassium chloride on water intake and urine volume in felines.

Twelve American domestic shorthair cats were randomly divided into 2 groups, 6 cats in each group. All cats were housed individually so that urine from each cat could be independently collected. One group was given a control food (Food 6) and the other group was fed the same food supplemented with additional potassium chloride (Food 7) for 21 days. The daily amount of food provided to each cat was adjusted for maintenance of body weight. Tap water was available to the cats at all times.

Food 6 comprised brewers rice, poultry by-product meal, corn gluten meal, calcium sulfate dihydrate, choline chloride, calcium carbonate, iodized salt, taurine, vitamin premix, mineral premix, animal fat, palatability enhancers, and potassium chloride (about 1.0% by weight of the food). Food 7 comprised the same ingredients supplemented with additional potassium chloride (about 3.4% by weight of the food).

Food intake and urine volume were recorded for each cat. Urine samples from all cats were collected during the last 5 days of the feeding period, and analyzed for urine volume, Cl and K. Results, averaged for all cats, are presented in Table 3.

TABLE 3
Results of feeding study comparing Foods 6 and 7 in felines

| Parameter | Food 6 | Food 7 |
|---|---|---|
| Body weight on day 21 (kg) | 4.57 | 5.02 |
| Food intake (g/day) | 47 | 54 |
| Urine volume (g/day) | 38.4 | 56.0 |
| K intake (g/day) | 0.51 | 1.05 |
| Urine K (g/day) | 0.31 | 0.44 |
| Cl intake (g/day) | 0.47 | 0.98 |
| Urine Cl (g/day) | 0.34 | 0.61 |

The cats receiving the potassium chloride-enriched food exhibited a 46% increase in urine volume by comparison with the cats receiving the control food (Food 6). This study provided further evidence that a major factor predisposing to urolithiasis in felines, namely low urine volume, could be ameliorated by feeding a potassium chloride-enriched food composition according to the invention.

### Example 4

A crossover study was conducted to investigate effect of increasing dietary potassium chloride on water intake and various urine parameters including urine volume and urine specific gravity in canines.

Eight beagles were randomly divided into 2 groups, 4 dogs in each group. All dogs were housed individually so that urine from each dog could be independently collected. One group was given a control food (Food 8) and the other group was fed the same food supplemented with additional potassium chloride (Food 9) for 10 days. The foods were then crossed between the two groups and fed for an additional 10 days. The daily amount of food provided to each dog was adjusted for maintenance of body weight. Deionized water was available to the dogs at all times.

Food 8 was a dry commercial canine food composition comprising yellow corn, poultry by-product meal, soybean meal, corn gluten meal, soybean mill run, compacted egg, calcium sulfate dihydrate, whole flaxseed, iodized salt, taurine, choline chloride, potassium chloride (about 0.2% by weight of the food), mineral premix, vitamin premix, animal fat, soybean oil, and palatability enhancers. Food 9 comprised the same ingredients supplemented with additional potassium chloride (about 2.3% by weight of the food).

Food intake and urine volume were recorded for each dog. Urine samples from all dogs were collected during the last 5 days of each study period (days 5-10 and 15-20), and analyzed for urine volume, specific gravity, P, Mg, Ca, Na and K. Results, averaged for all 8 dogs, are presented in Table 4.

TABLE 4
Results of feeding study comparing Foods 8 and 9 in canines

| Parameter | Food 8 | Food 9 |
|---|---|---|
| Food intake (g/day) | 257 | 268 |
| Dry matter intake (g/day) | 240 | 251 |
| Water intake (g/day) | 542 | 749 |
| Total water intake, including from food (g/day) | 560 | 766 |
| Total water intake/dry matter intake | 2.4 | 3.1 |
| Urine volume (g/day) | 229 | 306 |
| Urine specific gravity | 1.030 | 1.025 |
| Urine P (ppm) | 1046 | 692 |
| Urine Mg (ppm) | 75 | 35 |
| Urine Ca (ppm) | 66 | 41 |
| Urine Na (ppm) | 1807 | 1461 |
| Urine K (ppm) | 4926 | 9604 |

After consuming the potassium chloride-enriched food, the dogs exhibited a 37% increase in total water intake, a 29% increase in ratio of water intake to dry matter intake, and a 34% increase in urine volume. Urine specific gravity was reduced from 1.030 to 1.025. Concentrations of P, Mg, Ca, and Na in urine were also reduced.

In summary, this study showed that factors predisposing to urolithiasis in canines, including low urine volume and high urine specific gravity, could be ameliorated by feeding a potassium chloride-enriched food composition according to the invention.

In the specification, there have been disclosed typical preferred embodiments of the invention and, although specific terms are employed, they are used in a generic and descriptive sense only and not for purposes of limitation, the scope of the invention being set forth in the claims. Obviously many modifications and variations of the invention are possible in light of the above teachings. It is therefore to be understood that within the scope of the appended claims the invention may be practiced otherwise than as specifically described.

What is claimed is:

1. A food composition suitable for preventing or treating urolithiasis in an animal comprising potassium in an amount equivalent to more than about 1% by weight potassium chloride.

2. The composition of claim 1 wherein the composition is a dry food.

3. The composition of claim 2 wherein the dry food is in the form of kibbles.

4. The composition of claim 1 wherein the potassium is present at least in part as one or more salts selected from the group consisting of potassium chloride, potassium citrate, potassium sulfate, and combinations thereof.

5. The composition of claim 1 wherein the potassium is present wholly or partly as potassium chloride.

6. The composition of claim 1 wherein the amount of potassium is equivalent to not more than about 20% by weight potassium chloride.

7. The composition of claim 1 wherein the amount of potassium is equivalent to about 2% to about 8% by weight potassium chloride.

8. The composition of claim 1 wherein the amount of potassium is equivalent to about 3% to about 5% by weight potassium chloride.

9. The composition of claim 1 wherein the composition is nutritionally adapted for feeding to an animal as a substantial part of the animal's diet.

10. A method for preventing or treating urolithiasis in an animal comprising feeding the animal a food composition comprising potassium in an amount equivalent to more than about 1% by weight potassium chloride.

11. The method of claim 10 wherein the animal is canine or feline.

12. The method of claim 10 wherein the amount of potassium in the composition is equivalent to not more than about 20% by weight potassium chloride.

13. The method of claim 10 wherein the amount of potassium in the composition is equivalent to about 2% to about 8% by weight potassium chloride.

14. The method of claim 10 wherein the amount of potassium in the composition is equivalent to about 3% to about 5% by weight potassium chloride.

15. The method of claim 10 wherein the composition is nutritionally adapted for feeding to an animal as a substantial part of the animal's diet.

16. The method of claim 10 wherein the animal is fed the composition for a period of at least about 5 days.

17. The method of claim 10 further comprising administering to the animal an anti-urolithiasis agent.

18. The method of claim 17 wherein the anti-urolithiasis agent is a drug or nutraceutical that produces a desirable urine pH or prevents or treats bacterial infection of the urinary tract.

19. A kit comprising a potassium supplement and one or more of:
(a) a base food nutritionally adapted for feeding to an animal as a substantial part of its diet;
(b) an anti-urolithiasis agent other than a potassium supplement;
(c) a diagnostic device for measuring urine pH; and
(d) a means for communicating information about or instructions for one or more of (i) using said diagnostic device; (ii) adjusting or controlling urine pH; (iii) adding the potassium supplement to the base food to provide a food composition comprising potassium in an amount equivalent to more than about 1% by weight potassium chloride; and (iv) promoting urinary wellness or preventing or treating urolithiasis in an animal.

20. The kit of claim 19 wherein the supplement comprises potassium chloride.

21. A means for communicating information about promoting urinary wellness or preventing or treating urolithiasis in an animal by feeding the animal a food composition comprising potassium in an amount equivalent to more than about 1% by weight potassium chloride, the means being selected from the group consisting of product labels, package inserts, brochures, handouts, advertisements, public announcements, audiotapes, videotapes, DVDs, CD-ROMs, computer readable chips, cards and disks, computer memory, web pages, and combinations thereof.

22. A method for promoting urinary wellness in an animal comprising feeding the animal a food composition comprising potassium in an amount equivalent to more than about 1% by weight potassium chloride.

23. A use of a composition that comprises potassium in an amount equivalent to more than about 1% by weight potassium chloride to prepare a medicament for preventing or treating urolithiasis in an animal.
